(19) United States
(12) Patent Application Publication
(10) Pub. No.: US 2025/0221916 A1
(43) Pub. Date: Jul. 10, 2025

(54) PHOTOCURABLE COMPOSITION FOR NAILS

(71) Applicant: THREEBOND CO., LTD., Tokyo (JP)

(72) Inventor: Natsumi HARADA, Tokyo (JP)

(21) Appl. No.: 18/848,748

(22) PCT Filed: Mar. 24, 2023
(86) PCT No.: PCT/JP2023/011932

§ 371 Date: Sep. 19, 2024

(30) Foreign Application Priority Data
Apr. 1, 2022 (JP) 2022-062050

(51) Int. Cl. A61K 8/87; A61K 8/35; A61K 8/81; A61Q 3/02

(52) U.S. Cl. CPC A61K 8/87; A61K 8/35; A61K 8/8152; A61Q 3/02; A61K 2800/81; A61K 2800/95

(57) ABSTRACT

A photocurable composition for a nail contains the following (A) to (D): component (A): a urethane-modified oligomer having two or more (meth)acryloyl groups in one molecule and a weight average molecular weight of 25,000 to 100,000; component (B): a monomer having two or more ether groups and only one (meth)acryloyl group in the molecule; component (C) (excluding the component (A)): a monomer as a component (c-1) and/or a component (c-2); component (c-1): a chain monomer having four or more (meth)acryloyl groups in one molecule; component (c-2): a monomer having two or more (meth)acryloyl groups and a cyclic structure in one molecule; and component (D): a photoinitiator.

### TECHNICAL FIELD

The present invention relates to a photocurable composition for a nail, and in particular to a photocurable composition for a natural nail.

### BACKGROUND ART

For photocurable compositions for a nail or artificial nail, so-called gel nail, the coating film deteriorates over a certain period of time after treatment, and thus in a case of treatment again, it is necessary to remove the old coating film. It is known that the coating film is peeled off by softening with a solvent such as acetone, or by softening with hot water as in Patent Literature 1. It is also known that a film-like sticker can be used as a gel nail.

### CITATION LIST

### Patent Literatures

Patent Literature 1: JP 2002-161025 A

### SUMMARY OF INVENTION

However, the inventor has noticed that the method for using a solvent or hot water, that is, the method for removing the cured coating film by softening the coating film with a solvent such as acetone and peeling it off, may cause damage to the natural nail and inflammation of the skin of the finger. The inventor also has noticed that in particular, if the old coating film cannot be removed in one time, it may be necessary to use the solvent or hot water multiple times, which may cause damage to the natural nail and inflammation of the skin of the finger. In addition, the inventor has found that when a film-like sticker is used as a gel nail, the sticker cannot follow the unevenness of the surface of the natural nail, thus arising such a problem of not matching the shape of the natural nail and peeling off from the natural nail in a short time.

Therefore, an object is to provide a photocurable composition that allows the cured product as a coating film formed by curing the photocurable composition applied to the nail, to be peeled off as a single piece without breaking, making it easy to remove the coating film, and has excellent durability such that the coating film does not peel off in daily life.

As a result of intensive investigation on achieving the above-described object, the present inventor has found a method for a photocurable composition that allows the cured product formed by curing the photocurable composition applied to a natural nail, to be peeled off as a single piece without breaking when removing the cured product, and thus completed the present invention.

The summary of the present invention will be described. A first embodiment of the present invention is a photocurable composition for a nail, comprising the following (A) to (D):
component (A): a urethane-modified oligomer having a weight-average molecular weight of 25,000 to 100,000 and having two or more (meth)acryloyl groups in one molecule,
component (B): a monomer having two or more ether groups and only one (meth)acryloyl group in the molecule,
component (C) (excluding the component (A)): a monomer as a component (c-1) and/or a component (c-2),
component (c-1): a chain monomer having four or more (meth)acryloyl groups in one molecule,
component (c-2): a monomer having two or more (meth)acryloyl groups and an isocyanurate ring in one molecule, and
component (D): a photoinitiator.

A second embodiment of the present invention is the photocurable composition for a nail according to the first embodiment, wherein 80 to 150 parts by mass of the component (B) is comprised with respect to 100 parts by mass of the component (A).

A third embodiment of the present invention is the photocurable composition for a nail according to the first embodiment, further comprising a monomer (excluding the component (B)) having only one (meth)acryloyl group and only one hydroxyl group in one molecule.

A fourth embodiment of the present invention is the photocurable composition for a nail according to the first embodiment, wherein 0.01 to 10 parts by mass of the component (C) is comprised with respect to 100 parts by mass of the component (A).

A fifth embodiment of the present invention is the photocurable composition for a nail according to the first embodiment, further comprising an ultraviolet absorber.

A sixth embodiment of the present invention is the photocurable composition for a nail according to the fifth embodiment, wherein the ultraviolet absorber is a hydroxybenzophenone derivative.

A seventh embodiment of the present invention is the photocurable composition for a nail according to the first embodiment, wherein durometer hardness of a coating film formed by curing the photocurable composition for a nail is E10 to E50 in an atmosphere at 25° C.

An eighth embodiment of the present invention is a base coat agent comprising the photocurable composition for a nail according to any one of the first to seventh embodiments.

A ninth embodiment of the present invention is a peeling method comprising peeling off a cured product formed by photocuring the photocurable composition for a nail according to any one of the first to seventh embodiments applied to a natural nail, from the natural nail as a single piece without breaking, without using any solvent or hot water.

### DESCRIPTION OF EMBODIMENTS

Hereinafter, the embodiments of the present invention will be described. The present invention is not limited to the following embodiments. In addition, unless otherwise specified in this description, operations and measurements of physical properties are performed under the conditions of room temperature (20° C. or more and 25° C. or less) and relative humidity of 40% RH or more and 50% RH or less. Further, “A and/or B” includes each of A and B and all combinations of one or more, and specifically means at least one of A and B, and refers to A, B, and a combination of A and B. In this description, “X to Y” is used to mean that the numerical values (X and Y) written before and after it are included as lower and upper limits, and means “X or more and Y or less”. In a case where multiple “X to Y” are described, for example, in a case where “X1 to Y1 or X2 to Y2” is described, disclosures with each numerical value as the upper limit, disclosures with each numerical value as the lower limit, and combinations of those upper and lower limits are all disclosed (that is, they serve as lawful grounds for amendment).

Specifically, amendments with X1 or more, amendments with Y2 or less, amendments with X1 or less, amendments with Y2 or more, amendments with X1 to X2, amendments with X1 to Y2, and the like must all be considered lawful.

One aspect of the present invention provides a photocurable composition for a nail, comprising the following (A) to (D): component (A): a urethane-modified oligomer having a weight average molecular weight of 25,000 to 100,000 and having two or more (meth)acryloyl groups in one molecule, component (B): a monomer having two or more ether groups and only one (meth)acryloyl group in the molecule, component (C) (excluding the component (A)): a monomer as a component (c-1) and/or a component (c-2), component (c-1): a chain monomer having four or more (meth)acryloyl groups in one molecule, component (c-2): a monomer having two or more (meth)acryloyl groups and an isocyanurate ring in one molecule, and component (D): a photoinitiator. Therefore, a photocurable composition can be provided that allows the cured product as a coating film formed by curing the photocurable composition applied to the natural nail, to be peeled off as a single piece without breaking, making it easy to remove the coating film, and has a high durability such that the coating film does not peel off from the natural nail in a short time.

The details of the present invention are described below. Component (A) that can be used in the present invention is a urethane-modified oligomer having a weight average molecular weight of 25,000 to 100,000 and having two or more (meth)acryloyl groups in one molecule. Component (A) is preferably liquid in an atmosphere of 25° C., and can be used if it has good compatibility with the following components (B) and (C) of the present invention.

A urethane-modified oligomer having two or more (meth)acryloyl groups in one molecule is known to be synthesized by forming a urethane bond with a polyether polyol or polyester polyol and a polyisocyanate, and then adding a compound having a hydroxyl group and a (meth)acryloyl group in the molecule or (meth)acrylic acid to an unreacted isocyanate group. In addition, during synthesis, the synthesis may be performed in the monomer described below (that is, the monomer means a (meth)acrylate compound that does not contain a urethane-modified oligomer structure. The monomer may be a monomer with two or more functionalities, but typically a monofunctional monomer.). Specific examples of the component (A) include, but are not limited to, MB-111 manufactured by Negami Chemical Industrial Co., Ltd. In one embodiment, the number of (meth)acryloyl groups in one molecule of the component (A) may be more than 10, but is preferably 6 or less, 4 or less, or 3 or less.

In one embodiment, one or more (meth)acryloyl groups in the component (A) are each included as a part of a (meth)acryloyloxy group, and preferably all of the (meth)acryloyl groups in the component (A) are each included as a part of a (meth)acryloyloxy group.

The weight average molecular weight of the component (A) is 25,000 to 100,000. In one embodiment, the weight average molecular weight of the component (A) is 30,000 or more, 40,000 or more, 50,000 or more, 55,000 or more, or 60,000 or more. In one embodiment, the weight average molecular weight of the component (A) is 90,000 or less, 80,000 or less, 75,000 or less, or 70,000 or less. The addition of the component (A) imparts toughness to the cured product, achieving peelability. On the other hand, regardless of the number of functional groups (particularly the number of (meth)acryloyl groups), the molecular weight of the monomer described below is preferably 400 or less, and adding a monomer having such a molecular weight reduces the viscosity of the composition, making treatment easier. Herein, the weight average molecular weight refers to a weight average molecular weight measured by gel permeation chromatography in terms of polystyrene.

In addition, the urethane-modified oligomer as the component (A) may be used singly or in combination of two or more types. If two or more types are used in combination, the content of the component (A) refers to the total amount.

The component (B) that can be used in the present invention is a monomer having two or more ether groups (that is, ether structures, referred to as “ether groups” in this description) and only one (meth)acryloyl group in the molecule. The molecular weight of the component (B) is preferably 150 to 1,000, more preferably 150 to 800, and still more preferably 150 to 400. Further, in order to improve the peelability, it is preferable that the glass transition temperature of the compound obtained by curing the component (B) singly is 10° C. or less, 5° C. or less, −30° C. or less, −40° C. or less, or −50° C. or less. In addition, it is preferable that the glass transition temperature of the compound obtained by curing the component (B) singly is −100° C. or more, −90° C. or more, or −80° C. or more.

Herein, in the present description, the glass transition temperature can be measured, for example, as follows. A photoinitiator is dissolved in the monomer, and ultraviolet light with an integrated light quantity of 30 KJ/m² is irradiated to prepare a plate-shaped, cylindrical, or dot-shaped cured product, and the glass transition temperature can then be confirmed by heating from −100° C. to 200° C. at a rate of 10° C./min using a dynamic mechanical analyzer (DMA), thermomechanical analyzer (TMA), or differential scanning calorimetry (DSC).

In one embodiment, the (meth)acryloyl group in the component (B) is included as part of a (meth)acryloyloxy group.

In addition, the component (B) is preferably a compound represented by a general formula 1, in which R¹ represents a hydrogen atom or a methyl group, R² represents a divalent alkylene group, R³ represents a monovalent alkyl group, and n, which is the number of ether groups, represents an integer. More preferably, R² is an alkylene group having 1 to 3 carbon atoms, R³ is an alkyl group having 1 to 3 carbon atoms, and n is 2 to 10 or n is 2 to 3. In one embodiment, R² is an alkylene group having 1 to 3 or 2 to 3 carbon atoms. In one embodiment, R³ is an optionally substituted alkyl group having 1 to 20 carbon atoms, 1 to 15 carbon atoms, 1 to 12 carbon atoms, 1 to 10 carbon atoms, 1 to 8 carbon atoms, 1 to 6 carbon atoms, 1 to 4 carbon atoms, 1 to 3 carbon atoms, or 1 to 2 carbon atoms; or an optionally substituted aryl group having 6 to 20 carbon atoms, 6 to 16 carbon atoms, 6 to 12 carbon atoms, or 6 to 8 carbon atoms, in which the substitution means that a hydrogen atom is replaced by a halogen atom; an alkyl group having 1 to 20 carbon atoms, 1 to 15 carbon atoms, 1 to 12 carbon atoms, 1 to 10 carbon atoms, 1 to 8 carbon atoms, 1 to 6 carbon atoms, 1 to 4 carbon atoms, 1 to 3 carbon atoms, or 1 to 2 carbon atoms; an alkoxy group having 1 to 20 carbon atoms, 1 to 15 carbon atoms, 1 to 12 carbon atoms, 1 to 10 carbon atoms, 1 to 8 carbon atoms, 1 to 6 carbon atoms, or 1 to 4 carbon atoms; and an aryl group having 6 to 20 carbon atoms, 6 to 16 carbon atoms, 6 to 12 carbon atoms, 6 to 10 carbon atoms, or 6 to 8 carbon atoms; or a combination thereof. In one embodiment, n is 2 or more, 3 or more, 4 or more, 5 or more, or 6 or more. In one embodiment, n is 9 or less, 8 or less, 7 or less, 6 or less, 5 or less, 4 or less, or 3 or less. The component (B) is liquid in an atmosphere of 25° C.

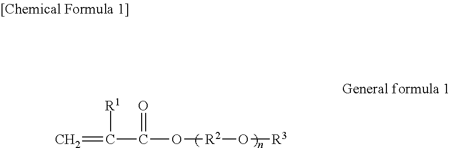

Specific examples of the component (B) include, but are not limited to, ethyl carbitol acrylate, methoxytriethylene glycol acrylate, methoxypolyethylene glycol acrylate, phenoxydiethylene glycol (meth)acrylate, phenoxytetraethylene glycol (meth)acrylate, nonylphenoxytetraethylene glycol (meth)acrylate, 2-ethylhexylpolyethylene glycol (meth)acrylate, and nonylphenylpolypropylene glycol (meth)acrylate, and these may be used singly or in combination. If two or more types are used in combination, the content of the component (B) refers to the total amount.

The component (B) is preferably contained in an amount of 80 to 150 parts by mass with respect to 100 parts by mass of the component (A). If the amount is 80 parts by mass or more, the coating film can be peeled off as a single piece without being damaged when peeled off, and if the amount is 150 parts by mass or less, durability can be maintained. In one embodiment, the component (B) is contained in an amount of 85 parts by mass or more, 90 parts by mass or more, 95 parts by mass or more, 100 parts by mass or more, 110 parts by mass or more, or 120 parts by mass or more with respect to 100 parts by mass of the component (A). In one embodiment, the component (B) is contained in an amount of 140 parts by mass or less, 130 parts by mass or less, 120 parts by mass or less, 110 parts by mass or less, or 100 parts by mass or less.

As a monomer other than the component (B) having only one (meth)acryloyl group in the molecule, other monofunctional (meth)acrylate monomers or (meth)acrylamide monomers can be used together with the component (B). In addition, these can be used singly or in combination of multiple types. If two or more types are used in combination, the content of monomers other than the component (B) having only one (meth)acryloyl group in the molecule refers to the total amount.

In one embodiment, the (meth)acryloyl group in a monomer having only one (meth)acryloyl group in the molecule other than the component (B) is included as part of a (meth)acryloyloxy group.

Other specific examples of monofunctional (meth)acrylate monomers include, but are not limited to, lauryl (meth)acrylate, methoxyethyl acrylate, phenoxyethyl (meth)acrylate, nonylphenoxyethyl (meth)acrylate, stearyl (meth)acrylate, tetrahydrofurfuryl (meth)acrylate, caprolactone-modified tetrahydrofurfuryl (meth)acrylate, cyclohexyl (meth)acrylate, dicyclopentanyl (meth)acrylate, isobornyl (meth)acrylate, benzyl (meth)acrylate, phenyl (meth)acrylate, 4-hydroxybutyl (meth)acrylate, 2-hydroxyethyl (meth)acrylate, 2-hydroxypropyl (meth)acrylate, N,N-dimethylaminoethyl (meth)acrylate, and N,N-diethylaminoethyl (meth)acrylate. From the viewpoint of adhesion to the natural nail, it is preferable to use a monomer that has only one (meth)acryloyl group and only one hydroxyl group in one molecule, and it is particularly preferable to use 2-hydroxyethyl (meth)acrylate or 2-hydroxypropyl (meth)acrylate.

According to one embodiment, the monomer having only one (meth)acryloyl group in the molecule other than the component (B) is a compound represented by the following general formula A.

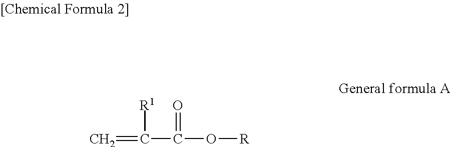

Herein, R¹ represents a hydrogen atom or a methyl group. In one embodiment, R may be an alkyl group having 1 to 20 carbon atoms, 3 to 18 carbon atoms, or 5 to 14 carbon atoms; or an alicyclic hydrocarbon group having 3 to 20 carbon atoms, 5 to 18 carbon atoms, or 6 to 14 carbon atoms. Herein, the alicyclic hydrocarbon group may be monocyclic or polycyclic, and may contain an aliphatic hydrocarbon group. In one embodiment, R may be a cyclohexyl group, an isobornyl group, an adamantyl group, a lauryl group, or a stearyl group.

In one embodiment, R may be a group containing only one ether group. In one embodiment, R is a group represented by —(CH₂)ₘ—O—R′, where m is 1 to 3, preferably 2 to 3, and R′ may be an alkyl group having 1 to 10 carbon atoms, 1 to 6 carbon atoms, 1 to 4 carbon atoms, 1 to 3 carbon atoms, or 1 to 2 carbon atoms; or an aryl group having 6 to 20 carbon atoms, 6 to 14 carbon atoms, 6 to 12 carbon atoms, or 6 to 10 carbon atoms, which is optionally substituted by an alkyl group having 1 to 10 carbon atoms, 1 to 6 carbon atoms, 1 to 4 carbon atoms, 1 to 3 carbon atoms, or 1 to 2 carbon atoms. In one embodiment, R may be a methoxyethyl group, a methoxypropyl group, an ethoxyethyl group, an ethoxypropyl group, a phenoxyethyl group, or a phenoxypropyl group.

In one embodiment, R may include a hydroxy group. In one embodiment, R may be a hydroxyalkyl group having 1 to 10 carbon atoms, 1 to 6 carbon atoms, 1 to 4 carbon atoms, or 1 to 3 carbon atoms. In one embodiment, R may be a hydroxymethyl group, a 1-hydroxyethyl group, a 2-hydroxyethyl group, a 1-hydroxypropyl group, a 2-hydroxypropyl group, a 3-hydroxypropyl group, or a 4-hydroxybutyl group.

Specific examples of (meth)acrylamide monomers include, but are not limited to, dimethyl(meth)acrylamide, (meth)acryloylmorpholine, and diethyl(meth)acrylamide. Specific examples of (meth)acrylamide monomers known in the present invention include, but are not limited to, DMAA, ACMO, and DEAA manufactured by KJ Chemicals Corporation.

The component (C) that can be used in the present invention is a chain monomer other than the component (A) that has four or more (meth)acryloyl groups in one molecule, which is a component (c-1), and/or a monomer that has two or more (meth)acryloyl groups and an isocyanurate ring in one molecule, which is a component (c-2). The cause is not clearly understood, but adding the component (C) reduces the risk of leaving scratches on the nail or leaving a cured product on the natural nail when peeled off. The component (C) is liquid in an atmosphere of 25° C. If two or more types are used in combination, the content of the component (C) refers to the total amount.

Specific examples of the (c-1) component include, but are not limited to, ditrimethylolpropane tetra(meth)acrylate, pentaerythritol tetra(meth)acrylate, dipentaerythritol penta(meth)acrylate, and dipentaerythritol hexa(meth)acrylate. Chain refers to a hydrocarbon chain, an alkylene oxide chain, or an ether chain, or the like, and the component (c-1) does not have a cyclic structure and has only a chain structure. In one embodiment, the number of (meth)acryloyl groups in one molecule of the component (c-1) is 5 or more, or 6 or more. In one embodiment, the number of (meth)acryloyl groups in one molecule of the component (c-1) is 10 or less, 8 or less, 6 or less, or 5 or less. In one embodiment, the molecular weight of the component (c-1) is 350 or more, 400 or more, 450 or more, or 500 or more. In one embodiment, the molecular weight of the component (c-1) is 1000 or less, 800 or less, 600 or less, 500 or less, or 400 or less.

In one embodiment, one or more (meth)acryloyl groups in the component (c-1) are each included as a part of a (meth)acryloyloxy group, and preferably all of the (meth)acryloyl groups in the component (c-1) are each included as a part of a (meth)acryloyloxy group.

In one embodiment, the component (c-1) is a compound represented by the following general formula 2 or general formula 3. In one embodiment, R^{4a} to R^{4f} in the general formula 2 are each independently a hydrogen atom, an optionally substituted alkyl group having 1 to 20 carbon atoms, 1 to 15 carbon atoms, 1 to 12 carbon atoms, 1 to 10 carbon atoms, 1 to 8 carbon atoms, 1 to 6 carbon atoms, 1 to 4 carbon atoms, 1 to 3 carbon atoms, or 1 to 2 carbon atoms, a hydroxyl group, or a (meth)acryloyloxy group, and at least four of R^{4a} to R^{4f} are (meth)acryloyloxy groups, and X¹ and X² in the general formula 2 are each independently an alkylene group having 1 to 3 carbon atoms or 1 to 2 carbon atoms. In one embodiment, R^{5a} to R^{5d} in the general formula 3 are (meth)acryloyloxy groups, and Y¹ to Y⁴ in the general formula 3 are each independently an alkylene group having 1 to 4 carbon atoms, 1 to 3 carbon atoms, or 1 to 2 carbon atoms.

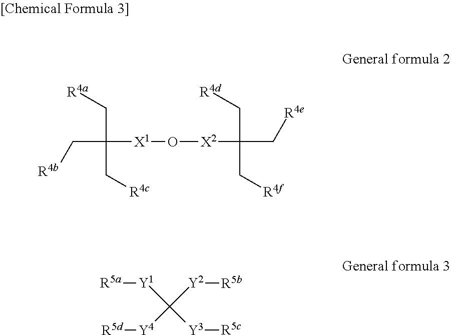

Specific examples of the component (c-2) include, but are not limited to, isocyanuric acid EO-modified di(meth)acrylate, and isocyanuric acid EO-modified tri(meth)acrylate, and these may be used in combination. The component (c-2) contains an isocyanurate ring as a cyclic structure and may contain a chain structure. Herein, EO means ethylene oxide. In one embodiment, the number of (meth)acryloyl groups in one molecule of the component (c-2) is 3 or more or 4 or more. In one embodiment, the number of (meth)acryloyl groups in one molecule of the component (c-2) is 6 or less, 4 or less, or 3 or less. In one embodiment, the molecular weight of the component (c-2) is 350 or more or 400 or more. In one embodiment, the molecular weight of the component (c-2) is 1000 or less, 800 or less, 600 or less, 500 or less, or 400 or less.

In one embodiment, one or more (meth)acryloyl groups in the component (c-2) are each included as a part of a (meth)acryloyloxy group, and preferably all of the (meth)acryloyl groups in the component (c-2) are each included as a part of a (meth)acryloyloxy group.

In one embodiment, the component (c-2) is a compound represented by the following general formula 4. In one embodiment, R^{6a} to R^{6c} in the general formula 4 are each independently a hydrogen atom, an optionally substituted alkyl group having 1 to 20 carbon atoms, 1 to 15 carbon atoms, 1 to 12 carbon atoms, 1 to 10 carbon atoms, 1 to 8 carbon atoms, 1 to 6 carbon atoms, 1 to 4 carbon atoms, 1 to 3 carbon atoms, or 1 to 2 carbon atoms, a hydroxyl group, or an acryloyloxy group, and at least two of R^{6a} to R^{6c} are acryloyloxy groups. In one embodiment, Z¹ to Z³ are each independently an alkylene group having 1 to 4 carbon atoms, 1 to 3 carbon atoms, or 1 to 2 carbon atoms.

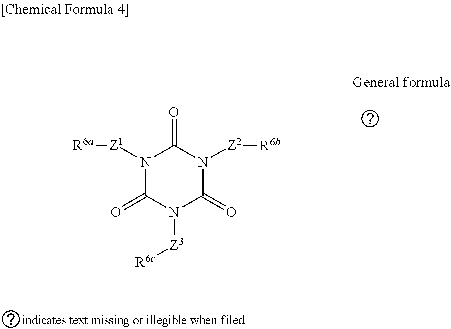

The component (C) is preferably contained in an amount of 0.01 to 10 parts by mass with respect to 100 parts by mass of the component (A). The component (C) is added in an amount of 0.01 part by mass or more, reducing the risk of scratching the natural nail or leaving a cured product on the natural nail when peeled off, and the component (C) is added in an amount of 10 parts by mass or less, allowing to suppress heat generation during curing. In one embodiment, the component (C) is contained in an amount of 0.02 parts by mass or more, 0.05 parts by mass or more, 0.1 parts by mass, 0.2 parts by mass, 0.5 parts by mass, 1.0 parts by mass or more, 1.5 parts by mass or more, or 2.0 parts by mass or more with respect to 100 parts by mass of the component (A). In one embodiment, the component (C) is contained in an amount of 9 parts by mass or less, 6 parts by mass or less, 4 parts by mass or less, 3 parts by mass or less, or 2.5 parts by mass or less with respect to 100 parts by mass of the component (A).

In addition, the total amount of the component (B), component (C), and other monofunctional (meth)acrylates is preferably 160 to 260 parts by mass with respect to 100 parts by mass of the component (A), which allows both peelability and durability to be achieved. In one embodiment, the total amount of the component (B), component (C), and other monofunctional (meth)acrylates is 180 parts by mass or more, 200 parts by mass or more, 220 parts by mass or more, or 230 parts by mass or more with respect to 100 parts by mass of the component (A). In one embodiment, the total amount of the component (B), component (C), and other monofunctional (meth)acrylates is 250 parts by mass or less, 240 parts by mass or less, 235 parts by mass or less, 230 parts by mass or less, or 225 parts by mass or less with respect to 100 parts by mass of the component (A).

The component (D) that can be used in the present invention is a photoinitiator (that is, a photopolymerization initiator, referred to as a “photoinitiator” in the present description). There are no limitations on the component (D) as long as it is a radical-based photoinitiator that generates radical species by energy rays such as visible light, ultraviolet light, X-rays, and electron beams.

In the present description, a visible light photoinitiator is a photoinitiator that has the strongest light absorption in the visible light region, and mainly refers to an acylphosphine oxide-based photopolymerization initiator that contains a phosphorus atom. Herein, visible light can be defined in JIS Z8120:2001. Specific examples thereof include, but are not limited to, 2,4,6-trimethylbenzoyl-diphenyl-phosphine oxide and bis(2,4,6-trimethylbenzoyl)-phenylphosphine oxide. Specific examples of non-visible light photoinitiators include, but are not limited to, acetophenones such as diethoxyacetophenone, 2-hydroxy-2-methyl-1-phenylpropan-1-one, benzyl dimethyl ketal, 4-(2-hydroxyethoxy)phenyl-(2-hydroxy-2-propyl) ketone, 1-hydroxycyclohexyl phenyl ketone, 2-methyl-2-morpholino (4-thiomethylphenyl) propan-1-one, 2-benzyl-2-dimethylamino-1-(4-morpholinophenyl) butanone, and 2-hydroxy-2-methyl-1-[4-(1-methylvinyl)phenyl]propanone oligomer; benzoins such as benzoin, benzoin methyl ether, benzoin ethyl ether, benzoin isopropyl ether, and benzoin isobutyl ether; benzophenones such as benzophenone, methyl o-benzoylbenzoate, 4-phenylbenzophenone, 4-benzoyl-4′-methyl-diphenyl sulfide, 3,3′,4,4′-tetra(t-butylperoxycarbonyl)benzophenone, 2,4,6-trimethylbenzophenone, 4-benzoyl-N,N-dimethyl-N-[2-(1-oxo-2-propenyloxy)ethyl]benzenemethanaminium bromide, and (4-benzoylbenzyl)trimethylammonium chloride; and thioxanthones such as 2-isopropylthioxanthone, 4-isopropylthioxanthone, 2,4-diethylthioxanthone, 2,4-dichlorothioxanthone, 1-chloro-4-propoxythioxanthone, and 2-(3-dimethylamino-2-hydroxy)-3,4-dimethyl-9H-thioxanthen-9-one mesochloride. In addition, a plurality of the components (D) can be used in combination. In one embodiment, the component (D) includes a combination of a visible light photoinitiator and a non-visible light photoinitiator. If two or more types are used in combination, the content of the component (D) refers to the total amount.

One to 25 parts by mass of the component (D) are added with respect to 100 parts by mass of the component (A), preferably 5 to 25 parts by mass, and more preferably 10 to 25 parts by mass. If the content of the component (D) is 1 part by mass or more, photocurability can be maintained. On the other hand, if the content of the component (D) is 25 parts by mass or less, storage stability can be maintained without thickening during storage. In addition, it is preferable that the visible light photoinitiator is contained in an amount of 30 to 70% by mass with respect to the entire component (D), and the cured product is less likely to yellow. More preferably, the visible light photoinitiator is contained in an amount of 35 to 60% by mass with respect to the entire component (D), and even more preferably, the visible light photoinitiator is contained in an amount of 40 to 50% by mass with respect to the entire component (D).

In one embodiment, the component (A) is contained in an amount of 25% by mass or more, 26% by mass or more, 27% by mass or more, 28% by mass or more, or 29% by mass or more, with respect to the entire photocurable composition for a nail. In one embodiment, the component (A) is contained in an amount of 55% by mass or less, 50% by mass or less, 45% by mass or less, 40% by mass or less, 38% by mass or less, 35% by mass or less, or 30% by mass or less, with respect to the entire photocurable composition for a nail.

In one embodiment, the total amount of the components (A), (B), (C), and (D) is 50 to 100% by mass, 60 to 90% by mass, or 65 to 80% by mass, with respect to the entire photocurable composition for a nail.

In one embodiment, the total amount of the component (A), the component (B), the component (C), other monofunctional (meth)acrylates, and the component (D) is 90 to 100% by mass, 95 to 99.9% by mass, 97 to 99.8% by mass, or 99.0 to 99.7% by mass with respect to the entire photocurable composition for a nail.

The present invention may contain additives such as coupling agents, inorganic or organic fillers, polythiol compounds, ultraviolet absorbers, colorants such as pigments and dyes, antioxidants, polymerization inhibitors, defoamers, leveling agents, and rheology control agents in appropriate amounts within the range that does not impair the characteristics of the present invention. The addition of these additives can provide a composition or a cured product thereof that is excellent in resin strength, adhesive strength, workability, storage stability, and the like.

In one embodiment, the total amount of the additives accounts for 0.1 to 15 parts by mass, 0.5 to 10 parts by mass, 1 to 8 parts by mass, 1.5 to 5 parts by mass, or 1.7 to 3 parts by mass, with respect to 100 parts by mass of the component (A).

In the present invention, a coupling agent can be added within a range that does not impair the characteristics of the present invention. Examples of the coupling agent include, but are not limited to, silane-based coupling agents having both an epoxy group, a vinyl group, an acryloyl group, or a methacryloyl group and a hydrolyzable silane group, polyorganosiloxanes having a phenyl group and a hydrolyzable silyl group, and/or polyorganosiloxanes having an epoxy group and a hydrolyzable silyl group. Specific examples of silane coupling agents include, but are not limited to, allyltrimethoxysilane, vinyltrimethoxysilane, 3-glycidoxypropyltrimethoxysilane, 3-methacryloxypropyltrimethoxysilane, 3-acryloxypropyltrimethoxysilane, and 3-chloropropyltrimethoxysilane.

In the present invention, fillers such as inorganic fillers and organic fillers can be added as appropriate within the range that does not impair the characteristics of the present invention. Adding a filler can adjust not only the viscosity and thixotropy but also the curability and toughness. Examples of inorganic fillers include, but are not limited to, alumina, silica, and amorphous silica. In contrast, examples of organic fillers include, but are not limited to, styrene filler, rubber filler, and core-shell acrylic filler. Examples of specific commercial products for silica include, but are not limited to, FUSELEX E-1 manufactured by TATSUMORI LTD. and AO-802 manufactured by Admafine Co., Ltd., and examples of specific commercial products for amorphous silica include, but are not limited to, the Aerosil series manufactured by NIPPON AEROSIL JAPAN CO., LTD., including 200 (untreated), R972 (treated with dimethyldichlorosilane), R976 (treated with dimethyldichlorosilane), RY200 (treated with dimethylsilicone), RX200 (treated with hexamethyldisilazane), and R800 (treated with octylsilane).

In the present invention, a polythiol compound can be added within a range that does not impair the characteristics of the present invention. The thiol compound is not particularly limited as long as it has two or more thiol groups, and only one type may be used, or two or more types may be used in combination. Specific examples of thiol compounds include, but are not limited to, aliphatic polythiol compounds, aromatic polythiol compounds, and polythiol compounds having a sulfide bond.

Examples of aliphatic polythiol compounds having two thiol groups include, but are not limited to, 1,2-ethanedithiol, 1,2-propanedithiol, 1,3-propanedithiol, 1,4-butanedithiol, 1,6-hexanedithiol, 1,7-heptanedithiol, 1,8-octanedithiol, 1,9-nonanedithiol, 1,10-decanedithiol, 1,12-dodecanedithiol, 2,2-dimethyl-1,3-propanedithiol, 3-methyl-1,5-pentanedithiol, 2-methyl-1,8-octanedithiol, 1,4-cyclohexanedithiol, 1,4-bis(mercaptomethyl)cyclohexane, 1,1-cyclohexanedithiol, 1,2-cyclohexanedithiol, bicyclo[2,2,1]hepta-exo-cis-2,3-dithiol, 1,1-bis(mercaptomethyl)cyclohexane, bis(2-mercaptoethyl)ether, ethylene glycol bis(2-mercaptoacetate), and ethylene glycol bis(3-mercaptopropionate).

Examples of aliphatic polythiol compounds having three thiol groups include, but are not limited to, 1,1,1-tris(mercaptomethyl)ethane, 2-ethyl-2-mercaptomethyl-1,3-propanedithiol, 1,2,3-propanetrithiol, trimethylolpropane tris(2-mercaptoacetate), trimethylolpropane tris(3-mercaptopropionate), and tris[(mercaptopropionyloxy)-ethyl]isocyanurate.

Examples of aliphatic polythiol compounds having four or more thiol groups include, but are not limited to, pentaerythritol tetrakis(2-mercaptoacetate), pentaerythritol tetrakis(3-mercaptopropionate), pentaerythritol tetrakis(3-mercaptobutyrate), and dipentaerythritol hexa-3-mercaptopropionate.

Examples of aromatic polythiol compounds include, but are not limited to, 1,2-dimercaptobenzene, 1,3-dimercaptobenzene, 1,4-dimercaptobenzene, 1,2-bis(mercaptomethyl)benzene, 1,3-bis(mercaptomethyl)benzene, 1,4-bis(mercaptomethyl)benzene, 1,2-bis(2-mercaptoethyl)benzene, 1,3-bis(2-mercaptoethyl)benzene, 1,4-bis(2-mercaptoethyl)benzene, 1,2-bis(2-mercaptoethyleneoxy)benzene, 1,3-bis(2-mercaptoethyleneoxy)benzene, 1,4-bis(2-mercaptoethyleneoxy)benzene, 1,2,3-trimercaptobenzene, 1,2,4-trimercaptobenzene, 1,3,5-trimercaptobenzene, 1,2,3-tris(mercaptomethyl)benzene, 1,2,4-tris(mercaptomethyl)benzene, 1,3,5-tris(mercaptomethyl)benzene, 1,2,3-tris(2-mercaptoethyl)benzene, 1,2,4-tris(2-mercaptoethyl)benzene, 1,3,5-tris(2-mercaptoethyl)benzene, 1,2,3-tris(2-mercaptoethyleneoxy)benzene, 1,2,4-tris(2-mercaptoethyleneoxy)benzene, 1,3,5-tris(2-mercaptoethyleneoxy)benzene, 1,2,3,4-tetramercaptobenzene, 1,2,3,5-tetramercaptobenzene, 1,2,4,5-tetramercaptobenzene, 1,2,3,4-tetrakis(mercaptomethyl)benzene, 1,2,3,5-tetrakis(mercaptomethyl)benzene, 1,2,4,5-tetrakis(mercaptomethyl)benzene, 1,2,3,4-tetrakis(2-mercaptoethyl)benzene, 1,2,3,5-tetrakis(2-mercaptoethyl)benzene, 1,2,4,5-tetrakis(2-mercaptoethyl)benzene, 1,2,3,4-tetrakis(2-mercaptoethyleneoxy)benzene, 1,2,3,5-tetrakis(2-mercaptoethyleneoxy)benzene, 1,2,4,5-tetrakis(2-mercaptoethyleneoxy)benzene, 2,2′-mercaptobiphenyl, 4,4′-thiobis-benzenethiol, 4,4′-dimercaptobiphenyl, 4,4′-dimercaptobibenzyl, 2,5-toluenedithiol, 3,4-toluenedithiol, 1,4-naphthalenedithiol, 1,5-naphthalenedithiol, 2,6-naphthalenedithiol, 2,7-naphthalenedithiol, 2,4-dimethylbenzene-1,3-dithiol, 4,5-Dimethylbenzene-1,3-dithiol, 9,10-anthracenedimethanethiol, 1,3-bis(2-mercaptoethylthio)benzene, 1,4-bis(2-mercaptoethylthio)benzene, 1,2-bis(2-mercaptoethylthiomethyl)benzene, 1,3-bis(2-mercaptoethylthiomethyl)benzene, 1,4-bis(2-mercaptoethylthiomethyl)benzene, 1,2,3-tris(2-mercaptoethylthio)benzene, 1,2,4-tris(2-mercaptoethylthio)benzene, 1,3,5-tris(2-mercaptoethylthio)benzene, 1,2,3,4-tetrakis(2-mercaptoethylthio)benzene, 1,2,3,5-tetrakis(2-mercaptoethylthio)benzene, and 1,2,4,5-tetrakis(2-mercaptoethylthio)benzene.

Examples of polythiol compounds having a sulfide bond include, but are not limited to, bis(2-mercaptoethyl) sulfide, bis(2-mercaptoethylthio) methane, 1,2-bis(2-mercaptoethylthio)ethane, 1,3-bis(2-mercaptoethylthio)propane, 1,2,3-tris(2-mercaptoethylthio)propane, tetrakis(2-mercaptoethylthiomethyl) methane, 1,2-bis(2-mercaptoethylthio)propanethiol, 2,5-dimercapto-1,4-dithiane, bis(2-mercaptoethyl)disulfide, 3,4-thiophenedithiol, 1,2-bis(2-mercaptoethyl)thio-3-mercaptopropane, and bis-(2-mercaptoethylthio-3-mercaptopropane) sulfide.

In addition, the polythiol compound preferably has a plurality of primary thiol groups (for example, —CH₂—CH₂—SH) or secondary thiol groups (for example, —CH₂—CHCH₃—SH) in the molecule. The effects on storage stability are different, and in consideration of stabilizing storage stability, it is preferable to have secondary thiol groups.

Specific examples of polythiol compounds having secondary thiol groups include, but are not limited to, pentaerythritol tetrakis(3-mercaptobutyrate), 1,4-bis(3-mercaptobutyryloxy)butane, 1,3,5-tris(3-mercaptobutyryloxyethyl)-1,3,5-triazine-2,4,6 (1H, 3H, 5H)-trione, trimethylolpropane tris(3-mercaptobutyrate), trimethylolethane tris(3-mercaptobutyrate), trimethylolpropane tris(3-mercaptobutyrate), and trimethylolethane tris(3-mercaptobutyrate). Examples of commercial products include, but are not limited to, PEMP manufactured by SAKAI CHEMICAL INDUSTRY CO., LTD., and the Karenz MT (registered trademark) series manufactured by Showa Denko KK, including PE1, BD1, and NR1.

In the present invention, an ultraviolet absorber can be added within a range that does not impair the characteristics of the present invention. Adding an ultraviolet absorber can suppress heat generation during photocuring. Particularly preferred is a phenolic compound of a general formula 5 having one phenol group in the molecule, as shown below. Herein, only one of R^{7a} to R^{7e} in the general formula 5 is an organic group conjugated with the aromatic ring of phenol, and R^{7a} to R^{7e} without the conjugated organic group are each independently a hydrogen atom or a chain organic group, and a chain organic group refers to a group that does not contain an aromatic ring or an alicyclic structure. Particularly preferred ultraviolet absorbers are hydroxybenzophenone derivatives. In one embodiment, the chain organic group can be an alkoxy group having 1 to 20, 2 to 15, or 3 to 10 carbon atoms.

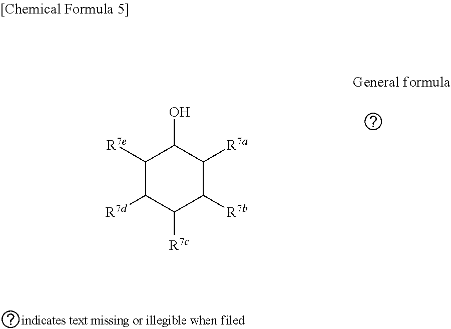

The organic group conjugated with the aromatic ring of the phenol is preferably a group represented by the formula 6, a group represented by a formula 7, or a group represented by a formula 8, or a group in which one or more substituents have been introduced into the aromatic ring in any one of the groups represented by formulas 6 to 8.

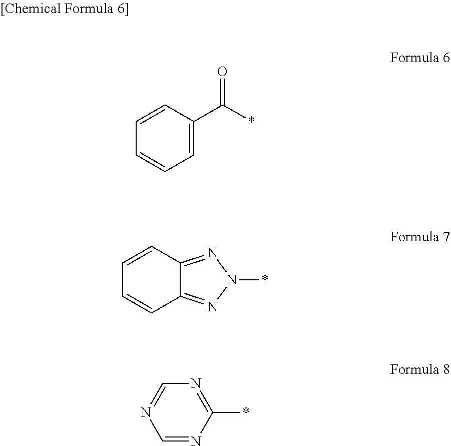 [SMILES: *C(=O)c1ccccc1 ; *n1nc2ccccc2n1 ; *c1ncncn1]

In formulas 6 to 8, * represents the bonding point with the compound (structure) of general formula 5.

A specific example of an ultraviolet absorber includes, but is not limited to, octabenzone.

In the present invention, a leveling agent may be added within a range that does not impair the characteristics of the present invention. Considering ease of application during treatment, those containing siloxane compounds or silicone resins are particularly preferable, and those containing modified polyorganosiloxanes are even more preferable. Specific examples thereof include, but are not limited to, polyether-modified polyorganosiloxanes, amine-modified polyorganosiloxanes, polydimethylsiloxanes, and siloxane-modified copolymers. Specific examples of leveling agents include, but are not limited to, the DISPARLON (registered trademark) LS series manufactured by Kusumoto Chemicals, Ltd., such as LS-430, LS-460, and LS-480; the KP series manufactured by Shin-Etsu Chemical Co., Ltd., such as KP-112, KP-323, KP-326, and KP-361; the TEGO (registered trademark) Glide series manufactured by TOMOE Engineering Co., Ltd., such as 100, 110, 410, 440, and 482; and the TEGO (registered trademark) Phobe series manufactured by TOMOE Engineering Co., Ltd., such as 1000, 1300, 1505, and 6600.

As used herein, the term “substituted” refers to the fact that a hydrogen atom is replaced with another atom or substituent. Unless otherwise specified, examples of the above atoms or substituents include, but are not limited to, halogen atoms; alkyl groups having 1 to 20 carbon atoms, 1 to 15 carbon atoms, 1 to 12 carbon atoms, 1 to 10 carbon atoms, 1 to 8 carbon atoms, 1 to 6 carbon atoms, 1 to 4 carbon atoms, 1 to 3 carbon atoms, or 1 to 2 carbon atoms; alkoxy groups having 1 to 20 carbon atoms, 1 to 15 carbon atoms, 1 to 12 carbon atoms, 1 to 10 carbon atoms, 1 to 8 carbon atoms, 1 to 6 carbon atoms, 1 to 4 carbon atoms, 1 to 3 carbon atoms, or 1 to 2 carbon atoms; acyloxy groups having 1 to 20 carbon atoms, 1 to 15 carbon atoms, 1 to 12 carbon atoms, 1 to 10 carbon atoms, 1 to 8 carbon atoms, 1 to 6 carbon atoms, 1 to 4 carbon atoms, 1 to 3 carbon atoms, or 1 to 2 carbon atoms; aryl groups having 6 to 20 carbon atoms, 6 to 16 carbon atoms, 6 to 12 carbon atoms, 6 to 10 carbon atoms, or 6 to 8 carbon atoms; or combinations thereof.

In the present description, examples of alkyl groups having 1 to 20 carbon atoms include, but are not limited to, methyl groups, ethyl groups, propyl groups, isopropyl groups, n-butyl groups, isobutyl groups, sec-butyl groups, tert-butyl groups, pentyl groups, isopentyl groups, neopentyl groups, hexyl groups, heptyl groups, octyl groups, nonyl groups, decyl groups, undecyl groups, lauryl groups, 2-ethylhexyl groups, tetradecyl groups, octadecyl groups, or icosyl groups.

In the present description, examples of alkoxy groups having 1 to 20 carbon atoms include, but are not limited to, methoxy groups, ethoxy groups, propoxy groups, isopropoxy groups, butoxy groups, pentyloxy groups, hexyloxy groups, 2-ethylhexyloxy groups, octyloxy groups, nonyloxy groups, decyloxy groups, undecyloxy groups, or dodecyloxy groups.

In the present description, examples of alicyclic hydrocarbon groups include, but are not limited to, cyclohexyl groups, methylcyclohexyl groups, dimethylcyclohexyl groups, norbornyl groups, methylnorbornyl groups, or isobornyl groups.

In the present description, the term “aryl group” refers to a cyclic substituent in which all elements have p-orbitals and these p-orbitals form conjugation, including monocyclic or condensed polycyclic (that is, rings sharing adjacent pairs of carbon atoms) functional groups. Examples of aryl groups having 1 to 20 carbon atoms include, but are not limited to, phenyl groups, naphthyl groups, anthracenyl groups, and biphenyl groups.

A method for preparing the photocurable composition for a nail of the present invention is not particularly limited, and a conventionally known method can be appropriately adopted. For example, the components (A), (B), (C), (D), and any optional components are weighed out in predetermined amounts and added to a stirring vessel in any order, either sequentially or simultaneously, and then mixed using a mixing means such as a planetary mixer, preferably with vacuum degassing. Preferably, the component (D) is added last. Adding the component (D) last can prevent the photopolymerization reaction from proceeding at an undesired stage. The production conditions are not particularly limited, but it is preferable to perform the process under light-shielded conditions. The mixing temperature is preferably 10 to 50° C., and the mixing time is preferably 0.1 to 5 hours.

The photocurable composition for a natural nail according to the present invention is liquid in an atmosphere of 25° C., and in consideration of ease of coat suitable for treatment, it is preferable that the viscosity is 5 to 35 Pas, 6 to 30 Pas, or 10 to 20 Pa·s.

A preferred method for using the photocurable composition for a nail according to one embodiment of the present invention will be described. In one embodiment of the present invention, before applying the photocurable composition for a nail to a natural nail (particularly human nail) or an artificial nail (that is, before performing treatment), the surface of the nail is sanded with a file or the like, and then dust, oil, moisture, and the like are removed with a solvent containing ethanol, acetone, or the like as a main component (solvents specifically for a nail are preferred). Then, the photocurable composition for a nail according to one embodiment of the present invention is applied to the nail with a brush or paintbrush such that the thickness of the composition before curing is 100 to 300 μm. The thickness of the composition before curing can be measured, for example, with a high-performance non-contact film thickness meter. Thereafter, photocuring is performed to provide a cured product (cured coating film). A commercially available UV lamp or LED lamp can be used as an irradiation device for curing. The irradiation time is preferably 15 to 120 seconds, and is preferably 20 to 70 seconds considering the effect on the fingers.

In one embodiment of the present invention, the cured gel nail is a laminate that may include a base coat (gel base; foundation) in contact with the nail or artificial nail, and, if necessary, a color coat and/or a top coat. The photocurable composition for a nail according to one embodiment of the present invention is particularly preferably used to form a base coat. That is, in one embodiment, a base coat agent containing the photocurable composition for a nail according to an aspect of the present invention is also provided. In such an embodiment, the base coat can be peeled off from the nail as a single coating film without breaking without using any solvent or hot water, and thus the entire laminate can be peeled off simply by peeling off the base coat, making it easy to perform the treatment again.

A cured product formed by photocuring the photocurable composition for a nail according to one embodiment of the present invention applied to a natural nail can be peeled off from the natural nail without using any solvent or hot water. That is, in one embodiment, there is provided a peeling method including peeling off a cured product formed by photocuring the photocurable composition for a nail according to an aspect of the present invention applied to a natural nail, from the natural nail without using any solvent or hot water. In one embodiment of the present invention, the temperature of the hot water is, for example, 35 to 47° C., 39 to 45° C., or 40 to 42° C.

In order to peeled off the coating film formed by photocuring the photocurable composition for a natural nail according to the present invention, from the natural nail as a single coating film without breaking, the hardness measured by an E-type durometer hardness measurement is preferably E10 to E50, and particularly preferably E20 to E40.

The embodiments of the present invention have been described in detail, and it is apparent that the same are illustrative and exemplary, not restrictive, and the scope of the present invention should be interpreted by the appended claims.

The present invention includes the following aspects and embodiments.

1. A photocurable composition for a nail, comprising the following (A) to (D):
component (A): a urethane-modified oligomer having a weight average molecular weight of 25,000 to 100,000 and having two or more (meth)acryloyl groups in one molecule,
component (B): a monomer having two or more ether groups and only one (meth)acryloyl group in the molecule,
component (C) (excluding component (A)): a monomer as a component (c-1) and/or a component (c-2),
component (c-1): a chain monomer having four or more (meth)acryloyl groups in one molecule,
component (c-2): a monomer having two or more (meth)acryloyl groups and an isocyanurate ring in one molecule, and
component (D): a photoinitiator;

2. The photocurable composition for a nail according to 1., wherein 80 to 150 parts by mass of the component (B) is comprised with respect to 100 parts by mass of the component (A);

3. The photocurable composition for a nail according to 1, or 2., further comprising a monomer (excluding the component (B)) having only one (meth)acryloyl group and only one hydroxyl group in one molecule;

4. The photocurable composition for a nail according to any one of 1. to 3., further comprising a compound represented by the following general formula A:

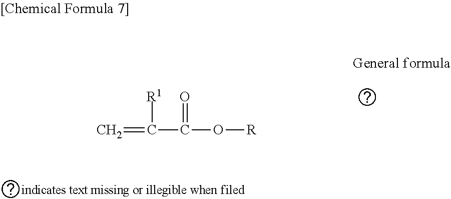

wherein R¹ is a hydrogen atom or a methyl group, and R is an alicyclic hydrocarbon group having 6 to 14 carbon atoms;

5. The photocurable composition for a nail according to any one of 1. to 4., wherein the component (D) includes a combination of a visible light photoinitiator and a non-visible light photoinitiator;

6. The photocurable composition for a nail according to any one of 1. to 5., wherein a weight average molecular weight of the component (A) is 50,000 to 100,000;

7. The photocurable composition for a nail according to any one of 1. to 6., wherein 0.01 to 10 parts by mass of the component (C) is comprised with respect to 100 parts by mass of the component (A);

8. The photocurable composition for a nail according to any one of 1. to 7., further comprising an ultraviolet absorber;

9. The photocurable composition for a nail according to 8., wherein the ultraviolet absorber is a hydroxybenzophenone derivative;

10. The photocurable composition for a nail according to any one of 1. to 9., wherein durometer hardness of a coating film formed by curing the photocurable composition for a nail is E10 to E50 in an atmosphere at 25° C.;

11. A base coat agent comprising the photocurable composition for a nail according to any one of 1. to 10.;

12. A peeling method comprising peeling off a cured product formed by photocuring the photocurable composition for a nail according to any one of 1. to 10. applied to a natural nail, from the natural nail without using any solvent or hot water.

### EXAMPLES

The present invention will be described in more detail with reference to Examples, but the present invention is not limited to these Examples.

### Examples 1 to 9 and Comparative Examples 1 to 9

The following components were prepared to provide a photocurable composition (hereinafter, the photocurable composition will also be referred to simply as the composition).

Component (A): a urethane-modified oligomer having a weight average molecular weight of 25,000 to 100,000 and having two or more (meth)acryloyl groups in one molecule
Urethane-modified oligomer with acryloyl groups and a weight average molecular weight of 65,000 (diluted to 40% by mass with isobornyl acrylate) (MB-111, manufactured by Negami Chemical Industrial Co., Ltd.)
Component (A′): Urethane-modified oligomer other than the component (A)
Urethane-modified oligomer with acryloyl groups and a weight-average molecular weight of 23,000 (diluted to 40% by mass with isobornyl acrylate) (MB-146, manufactured by Negami Chemical Industrial Co., Ltd.)
Urethane-modified oligomer with acryloyl groups and a weight-average molecular weight of 6,000 (Excellor RUA-075, manufactured by Asia Industry Co., Ltd.)
A urethane-modified oligomer with acryloyl groups and a weight-average molecular weight of 11,000 (NK Oligo UA-1138P, manufactured by SHIN-NAKAMURA CHEMICAL CO, LTD.)

Component (B): a monomer with two or more ether groups and only one (meth)acryloyl group in the molecule
Ethyl carbitol acrylate (molecular weight: 188) (glass transition temperature: −67° C.) (Viscoat #190, manufactured by Osaka Organic Chemical Industry Co., Ltd.)
Methoxy polyethylene glycol acrylate (molecular weight: 470) (glass transition temperature: unknown) (MPE400A, manufactured by OSAKA ORGANIC CHEMICAL INDUSTRY LTD.)

Other monofunctional monomers: monofunctional (meth)acrylate monomers other than the component (B)
2-hydroxypropyl methacrylate (glass transition temperature: −7° C.) (HPMA, manufactured by NIPPON SHOKUBAI CO., LTD.)
Phenoxyethyl acrylate (glass transition temperature: 2° C.) (Viscoat #192, manufactured by OSAKA ORGANIC CHEMICAL INDUSTRY LTD.)
Isobornyl acrylate (glass transition temperature: 97° C.) (IBXA, manufactured by OSAKA ORGANIC CHEMICAL INDUSTRY LTD.)
Lauryl acrylate (glass transition temperature: −23° C.) (L-A, manufactured by OSAKA ORGANIC CHEMICAL INDUSTRY LTD.)
Methoxyethyl acrylate (molecular weight: 130) (glass transition temperature: −50° C.) (2-MTA, manufactured by OSAKA ORGANIC CHEMICAL INDUSTRY LTD.)

Component (C): a monomer as a component (c-1) and/or a component (c-2)

Component (c-1): a chain monomer with 4 or more (meth)acryloyl groups in one molecule
Dipentaerythritol hexaacrylate (DPHA, DAICEL-ALLNEX LTD.)

Component (c-2): a monomer with 2 or more (meth)acryloyl groups and isocyanurate rings in one molecule
Caprolactone-modified tris-(2-acryloxyethyl) isocyanurate (NK Ester A9300-1CL, manufactured by SHIN-NAKAMURA CHEMICAL CO, LTD.)
Isocyanuric acid EO-modified di- and tri-acrylates (diacrylate: 35% by mass) (M-313, TOAGOSEI CO., LTD.)

Component (C′): monomers other than the component (C)
Tricyclodecane dimethanol diacrylate (Light acrylate DCP-A, KYOEISHA CHEMICAL Co., LTD.)
Trimethylolpropane triacrylate (A-TMPT, SHIN-NAKAMURA CHEMICAL CO, LTD.) Component (D): photoinitiator
1-Hydroxycyclohexyl phenyl ketone (non-visible light photoinitiator) (Suncure 84, manufactured by Chemark)
2,4,6-trimethylbenzoyl-diphenyl-phosphine oxide (visible light photoinitiator) (LUCIRIN TPO, manufactured by BASF)
   Other components
Octabenzone (reagent)
Polyether-modified silicone (LS-480, manufactured by Kusumoto Chemicals, Ltd.).

### [Preparation of Photocurable Composition]

A composition was prepared by weighing out a component (A), component (B), and/or other monofunctional (meth)acrylate monomer, component (C) (or component (C′)), and other components into a mixing vessel, and then stirring with a planetary mixer was performed with vacuum degassing for 30 minutes at room temperature under dark conditions. Then, a component (D) was weighed out and added to the mixing vessel, and stirring was performed for 30 minutes at room temperature with a light shield. Detailed preparation amounts are shown in Table 1 (Tables 1-1 to 1-3), and all values are expressed in parts by mass. The total of the component (B), other monofunctional (meth)acrylate monomer, and the component (C) (or component (C′)) is also shown in Table 1. The viscosity of the composition was also measured by the following method, and is shown in Table 1.

### [Viscosity Measurement]

0.5 ml of the composition was taken and dispensed into a measuring cup. Viscosity measurement was performed using an EHD viscometer (manufactured by Toki Sangyo Co., Ltd.) under the following conditions. The result was taken as “viscosity (Pas)”. Considering the treatment, a viscosity of 5 to 35 Pas is preferable.

Cone rotor: 3°×R14

Rotation speed: 100 rpm

Measurement time: 3 minutes

Measurement temperature: 25° C. (temperature controlled by a thermostatic bath)

TABLE 1-1

| Component | Raw material | Example 1 | Example 2 | Example 3 | Example 4 | Example 5 | Example 6 |
|---|---|---|---|---|---|---|---|
| Component (A) | MB-111 (oligomer | 100 | 100 | 100 | 100 | 100 | 100 |
| | component) | | | | | | |
| | MB-146 (oligomer | | | | | | |
| | component) | | | | | | |
| | RUA-075 | | | | | | |
| | UA-1138P | | | | | | |
| Component (B) | #190 | 123 | 123 | 123 | 123 | 123 | 107 |
| | MPE400A | | | | | | |
| Other | L-A | | | | | | |
| monofunctional | HPMA | 32 | 32 | 32 | 32 | 32 | 32 |
| monomers | #192 | | | | | | 16 |
| | 2-MTA | | | | | | |
| | IBXA | | | | | | |
| | IBXA (derived | 68 | 68 | 68 | 68 | 68 | 68 |
| | from component | | | | | | |
| | (A)) | | | | | | |
| Component (c-1) | DPHA | 2.3 | 2.3 | | | | 2.3 |
| Component (c-2) | 9300-1CL | | | 2.3 | | 6.5 | |
| | M-313 | | | | 2.3 | | |
| Component (C′) | DCPA | | | | | | |
| | TMPT | | | | | | |
| Component (D) | 84 | 9.7 | 9.7 | 9.7 | 9.7 | 9.7 | 9.7 |
| | TPO | 8.1 | 8.1 | 8.1 | 8.1 | 8.1 | 8.1 |
| Other | Oxybenzone 12 | 1.6 | 1.6 | 1.6 | 1.6 | 1.6 | 1.6 |
| components | LS-480 | 0.2 | | | | | 0.2 |

| | | | | | | |
|---|---|---|---|---|---|---|
| Total | 344.5 | 344.3 | 344.3 | 344.3 | 348.5 | 344.5 |
| Total of component (B), other | 225 | 225 | 225 | 225 | 229 | 225 |
| monofunctional monomers, and | | | | | | |
| component (C) (or component | | | | | | |
| (C′)) | | | | | | |
| Viscosity | 12.3 | 12.3 | 12.4 | 12.4 | 10.8 | 13.8 |

TABLE 1-2

| | | | | | Comparative | Comparative | Comparative |
|---|---|---|---|---|---|---|---|
| Component | Raw material | Example 7 | Example 8 | Example 9 | Example 1 | Example 2 | Example 3 |
| Component (A) | MB-111 | 100 | 100 | 100 | 100 | 100 | 100 |
| | (oligomer | | | | | | |
| | component) | | | | | | |
| | MB-146 | | | | | | |
| | (oligomer | | | | | | |
| | component) | | | | | | |
| | RUA-075 | | | | | | |
| | UA-1138P | | | | | | |
| Component (B) | #190 | 97 | | 90 | | 90 | 123 |
| | MPE400A | | 123 | | | | |
| Other | L-A | | | | 133 | | |
| monofunctional | HPMA | 32 | 32 | 32 | | 32 | 32 |
| monomers | #192 | 26 | | | | 32 | |
| | 2-MTA | | | 32 | | | |
| | IBXA | | | | 33 | | |
| | IBXA (derived | 68 | 68 | 68 | 67 | 68 | 68 |
| | from | | | | | | |
| | component | | | | | | |
| | (A)) | | | | | | |
| Component (c-1) | DPHA | 2.3 | 2.3 | 2.3 | | | |
| Component (c-2) | 9300-1CL | | | | | | |
| | M-313 | | | | | | |
| Component (C′) | DCPA | | | | | | |
| | TMPT | | | | | | |
| Component (D) | 184 | 9.7 | 9.7 | 9.7 | 10.0 | 9.7 | 9.7 |
| | TPO | 8.1 | 8.1 | 8.1 | 8.3 | 8.1 | 8.1 |
| Other | Oxybenzone 12 | 1.6 | 1.6 | 1.6 | | 1.6 | 1.6 |
| components | LS-480 | 0.2 | 0.2 | 0.2 | | | |

| | | | | | | |
|---|---|---|---|---|---|---|
| Total | 344.5 | 344.5 | 344.5 | 351.6 | 342.0 | 342.0 |
| Total of component (B), | 225 | 225 | 225 | 233 | 223 | 223 |
| other monofunctional | | | | | | |
| monomers, and component (C) | | | | | | |
| (or component (C′)) | | | | | | |
| Viscosity | 14.1 | 30.7 | 10.9 | 6.7 | 14.5 | 12.1 |

TABLE 1-3

| | Raw | Comparative | Comparative | Comparative | Comparative | Comparative | Comparative |
|---|---|---|---|---|---|---|---|
| Component | material | Example 4 | Example 5 | Example 6 | Example 7 | Example 8 | Example 9 |
| Component (A) | MB-111 | 100 | 100 | | | | 100 |
| | (oligomer | | | | | | |
| | component) | | | | | | |
| | MB-146 | | | 100 | | | |
| | (oligomer | | | | | | |
| | component) | | | | | | |
| | RUA-075 | | | | 100 | | |
| | UA-1138P | | | | | 100 | |
| Component (B) | #190 | 123 | 123 | 64 | 29 | 29 | |
| | MPE400A | | | | | | |
| Other | L-A | | | | | | |
| monofunctional | HPMA | 32 | 32 | 26 | 14 | 14 | 32 |
| monomers | #192 | | | | | | |
| | 2-MTA | | | | | | 123 |
| | IBXA | | | | | | |
| | IBXA | 68 | 68 | 67 | | | 68 |
| | (derived | | | | | | |
| | from | | | | | | |
| | component | | | | | | |
| | (A)) | | | | | | |
| Component (c-1) | DPHA | | | | | | 2.3 |
| Component (c-2) | 9300-1CL | | | | | | |
| | M-313 | | | | | | |
| Component (C′) | DCPA | | 2.3 | | | | |
| | TMPT | 2.3 | | | | | |
| Component (D) | 184 | 9.7 | 9.7 | 7.7 | 4.3 | 4.3 | 9.7 |
| | TPO | 8.1 | 8.1 | 6.4 | 3.6 | 3.6 | 8.1 |
| Other | Oxybenzone | 1.6 | 1.6 | | 0.7 | 0.7 | 1.6 |
| components | 12 | | | | | | |
| | LS-480 | | | | | | 0.2 |

| | | | | | | |
|---|---|---|---|---|---|---|
| Total | 344.3 | 344.3 | 270.5 | 151.5 | 151.5 | 344.5 |
| Total of component (B), | 225 | 225 | 156 | 43 | 43 | 225 |
| other monofunctional | | | | | | |
| monomers, and component | | | | | | |
| (C) (or component (C′)) | | | | | | |
| Viscosity | 12.3 | 12.2 | 10.4 | 9.5 | 10.2 | 7.8 |

Durometer hardness measurements were performed on Examples 1 to 9 and Comparative Examples 1 to 9. The results are summarized in Table 2.

### [Durometer Hardness Measurement]

A cured product of the composition was prepared into a square shape with a thickness of 10 mm and a smooth measurement surface. Hardness measurements were performed in accordance with JIS K 6253-3:2012 using a Shore E hardness tester under the following measurement conditions. The cured product was placed on the measurement table in an atmosphere of 25° C., and while keeping the pressure reference surface of the durometer horizontal to the sample surface, quickly pressing was performed with a force of 1 kgf on the measurement surface without shocking, whereby the pressure reference surface and the sample were in close contact. After the operation was completed, the maximum indication on the pointer was read as “hardness (unitless)” within 3 seconds as a general rule. In order for the coating film to be peeled off as a single piece without breaking, the hardness is preferably E10 to E50, and more preferably E20 to E40.

For Examples 1 to 9 and Comparative Examples 1 to 9, the treatment was performed by the following procedure for ten fingers of a subject, which were then left for three hours, after which the peelability and the condition of the natural nail after peeling were checked. The results are summarized in Table 2.

Step 1: dust, oil, moisture, and the like were removed from the natural nail with a solvent for a nail, containing ethanol as main component.

Step 2: a brush was used to form a 100 to 300 μm thick coating film on the natural nail before curing.

Step 3: the composition was irradiated with a nail LED lamp (rated voltage: 240V 50 to 60 Hz, power consumption: 30 W, wavelength: 365 to 410 nm) for 30 seconds to cure the composition.

### [Checking Peelability]

The interface between the natural nail and the coating film was hooked with the blade of a cutter to peel off the coating film from the edge, and visual inspection was performed according to the following evaluation criteria to determine “peelability”.

### Evaluation Criteria

Favorable: the coating film was peeled off as a single piece on the nails of all of the 10 fingers that were treated.

Poor: the coating film was peeled off with breaking.

[Checking Condition of Nail after Peeling]

After peeling off the coating film according to the peelability check, the condition of the natural nail in the peeled area was checked according to the following evaluation criteria to determine “natural nail condition”.

### Evaluation Criteria

Favorable: there were no scratches on the natural nails of all of the 10 fingers that were treated, and no residue of cured product was left.

Poor: there were scratches on the natural nail or residue of cured product on the nail.

TABLE 2

| Test item | Example 1 | Example 2 | Example 3 | Example 4 | Example 5 | Example 6 |
|---|---|---|---|---|---|---|
| Hardness | E30 | E30 | E30 | E30 | E30 | E30 |
| Peelability | Favorable | Favorable | Favorable | Favorable | Favorable | Favorable |
| Natural nail | Favorable | Favorable | Favorable | Favorable | Favorable | Favorable |
| condition | | | | | | |

| | | | | Comparative | Comparative | Comparative |
|---|---|---|---|---|---|---|
| Test item | Example 7 | Example 8 | Example 9 | Example 1 | Example 2 | Example 3 |
| Hardness | E31 | E30 | E32 | E32 | E31 | E28 |
| Peelability | Favorable | Favorable | Favorable | Favorable | Favorable | Poor |
| Natural nail | Favorable | Favorable | Favorable | Poor | Poor | Favorable |
| condition | | | | | | |

| | Comparative | Comparative | Comparative | Comparative | Comparative | Comparative |
|---|---|---|---|---|---|---|
| Test item | Example 4 | Example 5 | Example 6 | Example 7 | Example 8 | Example 9 |
| Hardness | E28 | E28 | E34 | E32 | E32 | E34 |
| Peelability | Poor | Poor | Poor | Poor | Favorable | Poor |
| Natural nail | Favorable | Favorable | Favorable | Poor | Poor | Poor |
| condition | | | | | | |

In the Examples and Comparative Examples, the hardness was in the range of E20 to E50, but the peelability and natural nail condition were both rated as “Favorable” in Examples 1 to 9, and it can be seen that the coating film was peeled off cleanly from the natural nail without breaking. The cause is unknown, but it is thought that this characteristic was manifested by combining the components (B) and (C) with the component (A). Meanwhile, in Comparative Example 1, which does not contain the components (B) and (C), Comparative Examples 2 to 8, which do not contain the component (C), and Comparative Example 9, which uses 2-MTA with a lower molecular weight than the component (B), instead of component (B), the peelability and natural nail condition were rated as “Poor” although the hardness was in the range of E10 to E50.

A durability test was performed for Examples 1 and 2. Using Examples 1 and 2, the treatment was performed on 10 fingers of a subject's hand using the following procedure.

Step 1: dust, oil, moisture, and the like are removed from the natural nail with a solvent for a nail, containing ethanol as main component.

Step 2: a coating film having 100 to 300 μm thick before curing is formed on the natural nail using a brush.

Step 3: the composition is irradiated with a nail LED lamp (rated voltage: 240 V 50 to 60 Hz, power consumption: 30 W, wavelength: 365 to 410 nm) for 30 seconds to cure the composition.

Step 4: a top coating of 100 to 300 μm thickness is formed, and irradiated with a nail LED lamp (rated voltage: 240 V 50-60 Hz, power consumption: 30 W, wavelength: 365 to 410 nm) for 30 seconds to cure the composition.

After the treatment, the subjects were asked to live their normal lives for 30 days, and the “durability” was visually checked according to the following evaluation criteria, and both Examples 1 and 2 were rated as “Favorable”.

### Evaluation Criteria

Favorable: the cured coating film had no damage or peeling.

Poor: the cured coating film had even the slightest damage or peeling.

In Examples 1 and 2, both the peelability and the condition of the natural nail are rated as “Favorable”, and the durability is also rated as “Favorable”. At first glance, a skilled person would assume that a favorable peelability leads to a deterioration of durability, but in the present invention, both properties are simultaneously exhibited.

### INDUSTRIAL APPLICABILITY

Using the photocurable composition for a nail according to the present invention, damage to the natural nail and inflammation of the skin of the fingers that occur when removing a cured coating film by softening the coating film with a solvent such as ethanol or acetone can be suppressed, and the coating film formed by curing the photocurable composition for a nail according to the present invention has favorable durability, and thus can be widely used as a base coat agent.

This application is based on Japanese Patent Application No. 2022-062050, filed on Apr. 1, 2022, the disclosure of which is incorporated herein by reference in its entirety.

1. A photocurable composition for a nail, comprising the following (A) to (D):
component (A): a urethane-modified oligomer having a weight average molecular weight of 25,000 to 100,000 and having two or more (meth)acryloyl groups in one molecule;
component (B): a monomer having two or more ether groups and only one (meth)acryloyl group in a molecule;
component (C) (excluding the component (A)): a monomer as a component (c-1) and/or a component (c-2);
component (c-1): a chain monomer having four or more (meth)acryloyl groups in one molecule;
component (c-2): a monomer having two or more (meth)acryloyl groups and an isocyanurate ring in one molecule; and
component (D): a photoinitiator.

2. The photocurable composition for a nail according to claim 1, wherein 80 to 150 parts by mass of the component (B) is comprised with respect to 100 parts by mass of the component (A).

3. The photocurable composition for a nail according to claim 1, further comprising a monomer (excluding the component (B)) having only one (meth)acryloyl group and only one hydroxyl group in one molecule.

4. The photocurable composition for a nail according to claim 1, further comprising g a compound represented by the following general formula A: 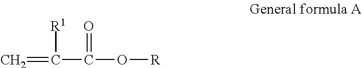
wherein R1 is a hydrogen atom or a methyl group, and R is an alicyclic hydrocarbon group having 6 to 14 carbon atoms.

5. The photocurable composition for a nail according to claim 1, wherein the component (D) includes a combination of a visible light photoinitiator and a non-visible light photoinitiator.

6. The photocurable composition for a nail according to claim 1, wherein a weight average molecular weight of the component (A) is 50,000 to 100,000.

7. The photocurable composition for a nail according to claim 1, wherein 0.01 to 10 parts by mass of the component (C) is comprised with respect to 100 parts by mass of the component (A).

8. The photocurable composition for a nail according to claim 1, further comprising an ultraviolet absorber.

9. The photocurable composition for a nail according to claim 8, wherein the ultraviolet absorber is a hydroxybenzophenone derivative.

10. The photocurable composition for a nail according to claim 1, wherein durometer hardness of a coating film formed by curing the photocurable composition for a nail is E10 to E50 in an atmosphere at 25° C.

11. A base coat agent comprising the photocurable composition for a nail according to claim 1.

12. A peeling method comprising peeling off a cured product formed by photocuring the photocurable composition for a nail according to claim 1 applied to a natural nail, from the natural nail without using any solvent or hot water.
